(19) United States
(12) Patent Application Publication
(10) Pub. No.: US 2005/0131278 A1
(43) Pub. Date: Jun. 16, 2005

(54) Endoscope

(75) Inventor: Jorg Dickopp, Reinbek (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 11/007,094

(22) Filed: Dec. 8, 2004

(30) Foreign Application Priority Data
Dec. 16, 2003 (DE) 103 58 817.5

(51) Int. Cl. A61B 17/04

(52) U.S. Cl. 600/107

(57) ABSTRACT

An endoscope includes an outer shaft with a substantially circular cross section, an inner shaft, which is arranged within the outer shaft and whose cross section comprises a rounded section and a straight section, which merge into one another at two corners, an optical system within the inner shaft, a flexible working instrument between the outer shaft and the straight section of the inner shaft, a deflecting lever, which is pivotally arranged at the distal end of the straight section of the inner shaft, at least one actuating rod, by means of which the deflecting lever may be pivoted by means of an actuating device arranged at the proximal end of the endoscope and at least one guide device, in which the actuating rod is arranged. The actuating rod is arranged within the inner shaft and the guide device is constituted by one of the corners and the optical system.

### BACKGROUND OF THE INVENTION

1. Field of the Invention

The invention relates to an endoscope such as a urethroscope, which is used for working in the urinary bladder.

2. Description of the Prior Art

Endoscopes of the type referred to above have a long outer shaft with a substantially round cross section. An inner shaft may be disposed within the outer shaft, the cross section of which comprises a rounded portion and a straight portion, which merge into one another via two corners. A flexible working instrument can be introduced between the outer shaft and the straight section of the inner shaft, the distal end of which projects, in the introduced state, out of the distal end of the outer shaft. The working instrument can be operated via an actuating device arranged on the proximal end of the outer shaft. Endoscopes of the type referred to above additionally have an optical system, which may be inserted into the inner shaft.

During urethroscopy, the endoscope is introduced into the urethra and pushed from there further into the urinary bladder. The doctor can then view the region to be examined with the aid of the optical system and, if required, undertake, for instance, medical intervention or remove a tissue sample by means of the working instrument. In endoscopes which are not of the type referred to above, the distal end of the working instrument extends straight from the outer shaft. This means that the doctor can only reach with the working instrument regions in the urinary bladder which are situated in straight alignment with respect to the outlet into the urethra. Regions in the urinary bladder, which are situated laterally of the outlet to the urethra, can, however, not be reached with an endoscope which is not of the type referred to above.

As a result of this, endoscopes of the type referred to above have, at the distal end of the flattened region of the inner shaft, a pivotable deflecting lever, which is also named an “Albarran lever”. The working doctor can laterally deflect the flexible working instrument projecting out of the outer shaft by means of the deflecting lever, whereby the possibility is created of also reaching lateral regions in the urinary bladder with the working instrument.

Pivoting of the deflecting lever is effected by means of at least one actuating rod, which is coupled to the actuating device disposed at the proximal end of the endoscope. The pivoting of the deflecting lever is effected by movement of the actuating rod in the pulling and pushing direction, which is produced by means of the actuating device. The actuating rod is a long, bar-shaped component, the rod cross section being very small in relation to the length of the rod as a result of the shortage of space in the outer shaft of the endoscope. This has the consequence that the actuating rod can deflect laterally under excessive pushing forces. As a result of this deflection, it can occur that there is inadequate force transmission to pivot the deflecting lever.

In order to prevent lateral deflection of the actuating rod, a guide device is provided in endoscopes of the type referred to above, in which the actuating rod is guided. It is known to use a tube as the guide device, the cross section of which is only insubstantially larger than the cross section of the actuating rod. Furthermore, the guide device is so constructed and arranged that the guide device surrounds the actuating rod from its distal end to its proximal end in order to prevent lateral deflection of it when transmitting thrust.

There is, however, the major disadvantage that fluids, such as blood and secretions etc., can accumulate during an examination in the very narrow space between the guide device and the actuating rod and it is very difficult to remove these liquids from it. Before the endoscope can be reused, thorough cleaning is, however, absolutely necessary.

DE 10009020 discloses an endoscope of the type referred to above, which mitigates this problem in that a plurality of guide sleeves are provided as the guide device, which are arranged behind one another in the axial direction on the outer surface of the inner shaft. The region to be cleaned is reduced in this manner but the problem of poor cleanability, however, basically remains since residues can also be located in the guide sleeves, which may be removed only with difficulty.

It is the object of the present invention to provide an endoscope which, on the one hand renders adequate guiding of the actuating rods possible and, on the other hand can be well cleaned.

### SUMMARY OF THE INVENTION

It is provided in accordance with the invention that the actuating rod is disposed within the inner shaft, the guide device being constituted by one of the corners and the optical system.

The guide device in the accordance with the invention is so constructed, as in the prior art, that it advantageously surrounds the actuating rod from its distal end to its proximal end so that deflection of the actuating rod is prevented over its entire length, when transmitting thrust. The guide device in accordance with the invention is, however, advantageously with respect to the prior art, capable of being taken apart such that the guide device may be very well cleaned.

For this purpose, the optical system, which constitutes a portion of the guide device in accordance with the invention, need merely be retracted in a manner which is in any event conventional. After the optical system has been withdrawn, the remaining portion of the guide device, namely the corner of the inner shaft and the actuating rod, are freely accessible within the inner shaft and may be easily reached, for instance by means of a brush or by washing, whereby thorough cleaning of the guide device is rendered possible.

A further advantage of the present invention is that the constitution of the guide device by the inner shaft and the optical system results in no additional component needing to be provided to constitute the guide device but instead components which are already present are so constructed that they assume responsibility for this function. The assumption of this function additionally offers the advantage that space can be saved in the cross sectional area of the endoscope, which is already very limited.

In known endoscopes of the type referred to above, the working instrument can catch on the actuating rods or the guide devices or guide sleeves during the introduction of the working instrument, whereby the introduction of the working instrument is frequently rendered difficult.

The present invention offers the advantage by comparison that the actuating rod and the guide device are concealed, that is to say guided within the inner shaft, so that catching on the working instrument is excluded from the outset. As a result of the transposition in accordance with the invention of the actuating rod from the outer cross sectional region, in which the working instrument is disposed, into the interior of the inner shaft, more space is also produced in the outer cross sectional region, whose construction is thus free for other purposes.

As already explained above, the distal end of the flexible working instrument can be laterally deflecting by means of the deflecting lever in order that the doctor can advance the distal end of the working instrument in a free and unimpeded matter to laterally situated regions of the urinary bladder. In accordance with an advantageous embodiment of the invention, the straight section of the inner shaft can have an integral inclined region at its distal end, formed by expansion of the shaft by a mandrel, on which the deflecting lever may be arranged. The distal end region of the working instrument is deflected with the aid of the inclined region into a first position during the introduction process. The distal end of the working instrument can then be deflected, if required, into a second, more strongly inclined position by means of the deflecting lever. The provision of the inclined region acts as a preparation for the subsequent deflection of the working instrument into a second position so that the force application necessary for this purpose is thus reduced.

In known endoscopes of the type referred to above, the inclined region is constituted by an inclined web, which is arranged and welded in a recess provided for this purpose at the distal end of the inner shaft. Various working steps are necessary for this purpose which are correspondingly cost intensive. By comparison, the construction of the inclined region in accordance with the invention results in a substantially more economical construction.

The invention will be explained below with reference to FIGS. 1 and 2 of the accompanying drawings.

### BRIEF DESCRIPTION OF THE DRAWINGS

FIG. 1 is a sectional view on the line 1-1 in FIG. 2 of the distal end of an endoscope in accordance with the invention

FIG. 2 is a sectional view on the line 2-2 in FIG. 1

### DESCRIPTION OF THE PREFERRED EMBODIMENT

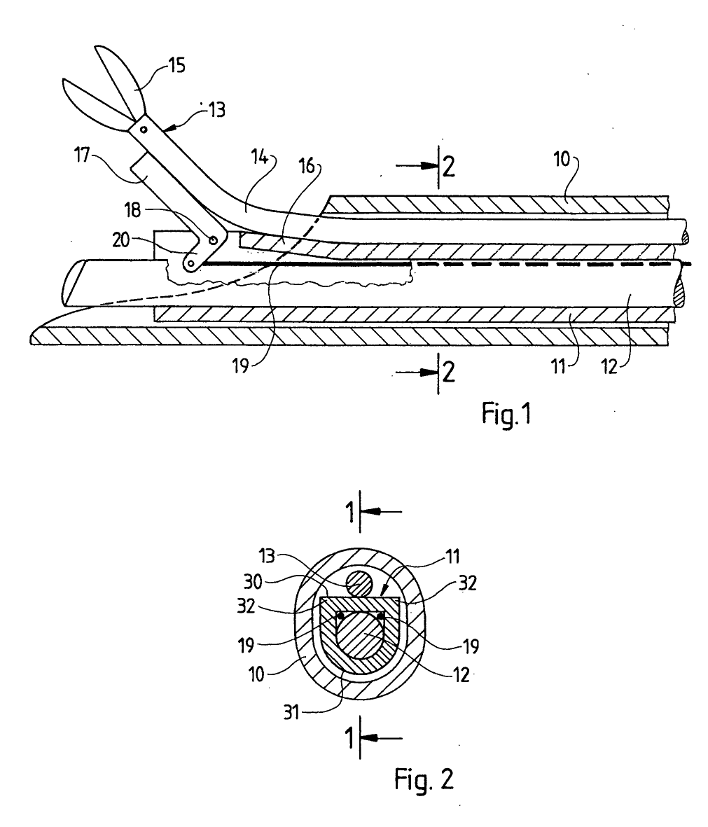

FIG. 1 is a sectional view of the distal end of an endoscope in accordance with the invention. The endoscope has an outer shaft 10, in which an inner shaft 11 is disposed. Retractably arranged in turn within the inner shaft 11 is an optical system 12, by means of which the doctor can view the region of the operation.

Introduced between the outer shaft 10 and the inner shaft 11 is a working instrument 13, which comprises a flexible shaft 14 and a working tool 15. At its distal end, the inner shaft 11 has an inclined region 16, by means of which the distal end of the working instrument 13 is easily deflected, as shown, during introduction into the endoscope from the direction of the shaft.

Arranged on the inclined region 16 is a deflecting lever 17. The deflecting lever 17 is pivotally connected at its proximal end to the inner shaft 11 by a pivotal shaft 18. The deflecting lever 17 is additionally pivotally connected at its proximal end via a laterally projecting arm 20 to an actuating rod 19, which is coupled to an actuating device (not shown) arranged at the proximal end of the endoscope.

In the introduction state, the deflecting lever 17 is aligned in the direction of the inner shaft 11. In order to be able to deflect the distal end of the working instrument 13 into the angled position, as shown in FIG. 1, the actuating rod 19 is pushed forwardly by the actuating device, whereby the deflecting lever 17 is pivoted upwardly about the pivotal shaft 18 and the distal end of the working instrument 13 is thus laterally deflected, as shown.

FIG. 2 shows, in cross section, the outer shaft 10 of the endoscope, which has a substantially round cross section. The inner shaft 11 and the working instrument 13 are arranged within the outer shaft 10.

The cross section of the inner shaft 11 is so shaped that it has a straight section 30 and a rounded section 31 and two corners 32, the transition from the straight to the rounded section occurring via respective corners 32.

The optical system 12 is disposed in the inner shaft. A respective actuating rod 19 is additionally disposed in each corner 32. As is clear in FIG. 2, the cross section of the inner shaft is so shaped that, when the optical system 12 is inserted, the two actuating rods 19 are closely surrounded by the two inner corners 32 of the inner shaft 11 and the optical system and thus enable guiding of the actuating rods 19 in this manner.

The optical system 12 can be withdrawn for cleaning purposes. After the optical system 12 has been pulled out, the remaining guide device, which is constituted by the two inner corners 32 of the inner shaft 11, and the two actuating rods 19 are freely accessible within the inner shaft 11. Cleaning can now be effected, e.g. by washing.

In a further exemplary embodiment, the endoscope in accordance with the invention can have only one actuating rod. The cross section of the inner shaft can be shaped in accordance with the first exemplary embodiment.

1. An endoscope including an outer shaft, which has a substantially round cross section, an inner shaft, which is disposed in the outer shaft and whose cross section comprises a rounded section and a straight section, which merge into one another at two corners, an optical system, which is disposed in the inner shaft, a flexible working instrument, which is disposed between the outer shaft and the straight section of the inner shaft, a deflecting lever, which is pivotally arranged at the distal end of the straight section of the inner shaft, at least one actuating rod, by means of which the deflecting lever may be pivoted by means of an actuating device arranged at the proximal end of the endoscope, and at least one guide device, in which the actuating rod is arranged, wherein the actuating rod is arranged within the inner shaft and the guide device is constituted by one of the corners and the optical system.

2. An endoscope as claimed in claim 1, characterised in that the straight section of the inner shaft has an integral inclined region at its distal end on which the deflecting lever is arranged.
